(19) United States
(12) Patent Application Publication
(10) Pub. No.: US 2005/0284486 A1
(43) Pub. Date: Dec. 29, 2005

(54) Devices and methods to reduce, ease, or eliminate snoring

(76) Inventor: Richard L. Martin, Mesquite, TX (US)

Correspondence Address: CRUTSINGER & BOOTH, 1601 ELM STREET, SUITE 1950, DALLAS, TX, 752014744, US

(21) Appl. No.: 10/878,082

(22) Filed: Jun. 28, 2004

(51) Int. Cl. A61C 3/00; A61F 5/56

(52) U.S. Cl. 128/848

(57) ABSTRACT

The device includes a band, wherein a forward portion of the band is adapted to be positioned across the mouth, and a rearward portion of the band is adapted to be positioned around the back of the head or neck. The device includes an air passage through at least a forward portion of the band and at least one remote air aperture located on the band remote from the mouth. In one aspect, the air passage has at least one mouth air aperture communicating with the air passage. In a second aspect, an insert is connected to the forward portion of the band, the insert adapted to be positioned to extend into the mouth between the tongue and hard palate but not so far as to contact the posterior pharynx. In a third aspect, the device also includes an air passage through at least a portion of the insert.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO MICROFICHE APPENDIX

Not applicable

### TECHNICAL FIELD

The devices and methods relate to reducing, easing, or eliminating snoring, sometimes referred to in the art as “anti-snoring” devices and methods.

### BACKGROUND OF THE INVENTION

Snoring is a common problem in that it can interfere with a person's own restful sleeping, and it can sometimes contribute to sleep apnea and other such breathing disorders. Of course, another common problem with snoring is the noise can be disturbing to others. It can disrupt sleeping arrangements and family relationships.

It is well known in the art that to snore is to breathe during sleep with a rough hoarse noise due to vibration of the uvula and the soft palate. The uvula is the pendent fleshy lobe in the middle of the posterior border of the soft palate. The soft palate is the membranous and muscular fold suspended from the posterior margin of the hard palate and partially separating the mouth cavity from the pharynx—called also velum.

The hard palate is a part of the human palate supported by the maxillary and palatine bones. The pharynx is the part of the alimentary canal between the cavity of the mouth and the esophagus that is in man a conical musculomembranous tube about four and a half inches long, continuous above the mouth and nasal passages, communicating through the eustachian tubes with the ears, and extending downward past the opening into the larynx to the lower border of the cricoid cartilage where it is continuous with the esophagus.

As used herein, the mouth is a: the opening through which food passes into the body of an animal, specifically, the orifice in the head of higher vertebrates bounded by the lips or jaws; b: the cavity bounded externally by the lips or jaws and internally by the pharynx or gullet that encloses in the typical vertebrate the tongue, gums, and teeth; the buccal cavity; c: the structures enclosing or lying within the mouth cavity regarded as a whole <the dog seized the bone in his mouth> <my mouth is sore>.

The above definitions are drawn from Webster's Third New International Dictionary, Unabridged. Merriam-Webster, 2002. This dictionary is hereby selected as the dictionary of reference for the purposes of describing and claiming this invention.

Snoring is a very common problem, and numerous efforts to address these problems have been made, but none of them is generally satisfactory.

U.K. Patent No. 633,247 issued Dec. 12, 1949 and having for named inventor Maureen Punnett discloses a device with an elastic mask covering the upper lip, mouth and chin which is held in place by two elastic bands, one under the ears to the back of the head, and one from check to cheek over the crown of the head. Through the center of the mask covering the mouth is a round aperture. Covering the aperture on the outside of the mask is a short tube, twice the diameter of the aperture. At the point between tube and mask is a rubber disc, which at one point on its circumference has a slight projection, which is trapped between the tube and mask; acting as a hinge for the rubber disc, allowing it to move within the tube during breathing. In operation, the disc covering the aperture over the mouth, allows free expulsion of breath but automatically closes on the intake of breath, thus forcing the wearer to inhale through the nose. U.K. Patent No. 633,247.

U.S. Pat. No. 3,312,217 issued Apr. 4, 1967 and having for named inventor W. S. McKinstry discloses a simplified device for very comfortably holding the mouth of a sleeping person in closed condition, to prevent snoring by such person, and a device that can be readily adjusted by the user to expeditiously compensate for different physical characteristics or dimensions existing between different people. U.S. Pat. No. 3,312,217, column 1, lines 19-24.

U.S. Pat. No. 4,270,531 issued Jun. 2, 1981 and having for named inventor Paul H. Blachly, describes an oropharyngeal airway tube and bite block assembly having means for connection of a ventrilatory reuscitator bag. The bite block provides an air-tight seal between the oropharyngeal airway tube and the gums, teeth, and lips of a patient which, in combination with conventional nostril occluding means, permits closed pulmonary ventilation of a patient without the need for a face mask nor endotracheal tube. The bite block properly locates and provides protection for the airway tube to provide an unobstructed air passage. U.S. Pat. No. 4,270,531, Abstract.

U.S. Pat. No. 5,046,512 issued Sep. 10, 1991 and having for named inventor John A. Murchie discloses an improved method and apparatus for treatment of snoring and apnea. The method provides the steps of regulating the flow of air through the mouth of the user to an extent comparable to the volume of air which flows through the user's nasal passages. The apparatus provides a device having a body portion sufficiently wide to separate the user's teeth and provided with an air passage comparable in area to the area of the user's nasal passages. In a preferred form of the invention the user is able to seal the air passage with his tongue to completely suppress the flow of air through the mouth and to control the turbulence of the air meeting the nasal inspiration. U.S. Pat. No. 5,046,512, Abstract.

U.S. Pat. No. 5,154,184 issued Oct. 13, 1992 and having for named inventor Ramiro M. Alvarez discloses an anti-snoring apparatus comprising receptacle structure configured for reception and retention of outer extent of the user's tongue; shield structure shaped to be received and retained on the tongue receptacle structure and positioned just forward of the user's lip or lips, and attachment structure for adjustably attaching the shield structure to the receptacle structure to permit selective adjustment of the position of the receptacle structure relative to the tongue forward extent whereby the tongue may be comfortably positioned and retained in the receptacle structure; whereby snoring is reduced as the tongue is brought forward, out of the mouth, and incremental forward movement of the shield will move the tongue further forward, with lessened airway obstruction. U.S. Pat. No. 5,154,184, Abstract.

U.S. Pat. No. 5,720,302 issued Feb. 24, 1998 and having for named inventor William A. Belfer discloses an anti-snoring device including a denture member for covering the lower teeth of the user and for maintaining the tongue in contact with the palate to prevent air flow from causing the palate to reverberate during mouth breathing; an extension member fixedly connected to the front of the denture member; and a flexible oral shield having an opening for slidably receiving the extension member and having at least one breathing hole. The flexible oral shield is worn outside the mouth and is conformable to the user's lips for preventing the lower jaw from drifting inferiorly and posteriorly during sleep. U.S. Pat. No. 5,720,302, Abstract.

U.S. Pat. No. 6,371,112 issued Apr. 16, 2002 and having for named inventor Noam Bibi discloses a self contained CPAP device. The device includes, a mouthpiece, a breathing tube, an inflatable body and a mask. The mouthpiece is insertable in a mouth of a patient and is attachable to the breathing tube which contains at least one bi-directional valve. The inflatable body functions to adjust an internal cross sectional area of the breathing tube and further serves to regulate air pressure within the patient's mouth. The mask is in fluid communication with the inflatable body and covers the nose of the patient so that exhaled air is routed thereto. Further disclosed is a system for prophylactic treatment of a breathing disorder which includes a sell contained CPAP device. Further disclosed is an improved method of preventing a breathing disorder by means of CPAP. The method includes the steps of inserting mouthpiece and allowing inhaled air to flow through a bi-directional valve contained within a breathing tube. The method also includes die steps of adjusting an internal cross sectional area of the breathing tube by means of an inflatable body which regulates an, air pressure within the mouth and covering a nose of the patient with a mask communicating which the inflatable body. The mask receives exhaled air and routes it to the nose of the patient. U.S. Pat. No. 6,371,112, Abstract.

U.S. Pat. No. 6,474,339 B1 issued Nov. 5, 2002 and having for named inventors Jacques Grosbois and Christian Michaud discloses a device for suppressing snoring and apnoea during seep. It is placed in the oro-pharynx and at the buccal cavity region during sleep. It comprises a flexible tube (1) arciform and transversally flattened, of adapted anatomical design, for being placed between the tongue (13) and the soft palate (15), down to at least the base of the tongue (14). A filter (2) is fixed to the external end of this tube. The filter can be removable. Thus, the air breathed in (10) moves freely from outside up to the larynx, without snoring or apnoea. A fixing system (24) is provided so that the tube is maintained well inside the mouth during sleep. U.S. Pat. No. 6,474,339B1, Abstract.

U.S. Pat. No. 6,675,804 B1 issued Jan. 13, 2004 and having for named inventor Alexander R. Pivovarov discloses an apparatus adapted for partial insertion within the user's mouth for preventing snoring, teeth grinding, and light forms of sleep apnea. The apparatus includes a multi-lobed tongue receiving structure, an undulating connector for connecting the multi-lobed structure to an inner lip plate, a hollow tube connecting the lip plate to a dome-shaped structure formed on an outer shield. The device is inserted within the oral cavity of the user in an operative configuration such that movement of the tongue is restrained within the multi-lobed structure, and the teeth clamp down upon the undulating connector with the lip plate positioned between the teeth and the inner portions of the upper and lower lips. As a result of proper application of the apparatus breathing at night is normalized, while snoring, grinding of the teeth, and apnea are prevented. U.S. Pat. No. 6,675,804B1, Abstract.

U.S. Patent Application Publication No. 2004/0016432 A1 published Jan. 29, 2004 and having for named inventors Harald Genger, Martin Baeke, and Hartmut Schneider discloses an anti-snoring device comprising a compressor and a nasal air cannula, the air compressed by the compressor being blown through the nasal air cannula into the nose of a sleeping person. The invention also relates to an optimized nasal air cannula for the anti-snoring device. U.S. Patent Application Publication No. 2004/0016432 A1, Abstract.

Several of these prior-art devices depend on keeping the mouth closed with a chin strap. Other types are mouth pieces, similar to pacifiers, that can easily fall out of the mouth. Some of the mouth piece devices are similar to teeth guards, that are too intrusive into the mouth. Some of these devices appear to be likely to cause or allow drooling from the mouth when laying so that the head is on its side. Some of the devices prevent sleeping on the side or face down.

Worse, several of these prior-art devices use tubes extending into the mouth and to the pharynx, which can cause a gag reflex. The normal gag reflex is a typical response to posterior pharynx tactile stimulation, which results in a gag, i.e., pharyngeal wall and velar contraction.

As can be seen from the foregoing survey representative of the art relating to anti-snoring devices, there has been and continues to be a long-felt need for a simple, comfortable, adjustable, and inexpensive solution to snoring and similar breathing problems. In addition, people even complain about the snoring of their dogs. Dogs, having a similar anatomy to humans, can also snore.

For normal, restful breathing, and particularly for normal sleeping, it is important to be able to inhale and exhale freely, without sense or feeling of constriction. It is also desirable to avoid drying out of the mouth, avoid causing an over abundance of saliva, and to avoid causing drooling. Further, it is desirable to allow a person to comfortably use an anti-snoring device while sleeping in any natural position.

### SUMMARY OF THE INVENTION

According to a first aspect of the invention, a device to reduce, ease, or eliminate snoring is provided. The device includes a band, wherein a forward portion of the band is adapted to be positioned across the mouth and a rearward portion of the band is adapted to be positioned around the back of the head or neck. The device includes an air passage through at least a portion of the length of the forward portion of the band. The air passage of the forward portion of the band has at least one mouth air aperture communicating with the air passage located on the forward portion of the band such that, when the forward portion of the band is positioned across the mouth, the air aperture is oriented toward the mouth. At least one remote air aperture communicating with the air passage located on the band remote from the mouth.

According to a second aspect of the invention, a device to reduce, ease, or eliminate snoring is provided, which is similar to a device according to a first aspect of the invention with the addition of an insert. The insert is connected to the forward portion of the band, and the insert is adapted to be positioned to extend into the mouth between the tongue and hard palate but not so far as to contact the posterior pharynx.

According to a third aspect of the invention, a device to reduce, ease, or eliminate snoring is provided. The device includes a band, wherein a forward portion of the band is adapted to be positioned across the mouth and a rearward portion of the band is adapted to be positioned around the back of the head or neck. The device also includes an insert, wherein the insert is connected to the forward portion of the band, and the insert is adapted to be positioned to extend into the mouth between the tongue and hard palate but not so far as to contact the posterior pharynx. According to this aspect of the invention, the device includes an air passage through at least a portion of the length of the forward portion of the band, wherein the air passage of the forward portion of the band has at least one remote air aperture communicating with the air passage located on the band remote from the mouth. The device also includes an air passage through at least a portion of the insert, wherein the air passage of the insert has at least one insert air aperture adapted to be positioned in the mouth and communicates with the air passage of the forward portion of the band.

These and further aspects and advantages of the invention will become apparent to persons skilled in the art from the following drawings and detailed description of presently most-preferred embodiments of the invention. These and further aspects of the invention are most advantageously and synergistically practiced together.

### BRIEF DESCRIPTION OF THE DRAWING

The accompanying drawings are incorporated into and form a part of the specification to illustrate several examples of the present inventions. These drawings together with the description serve to explain the principles of the inventions. The drawings are only for illustrating preferred and alternative examples of how the inventions can be made and used and are not to be construed as limiting the inventions to the illustrated and described examples. The various advantages and features of the present inventions will be apparent from a consideration of the drawings in which:

FIG. 1 is a view of a device to reduce, ease, or eliminate snoring according to the first aspect of the invention;

FIG. 2 is a view of a device to reduce, ease, or eliminate snoring according to the third aspect of the invention;

FIG. 3 is a view of a device to reduce, ease, or eliminate snoring according to the second and third aspects of the invention, and illustrating an alternative connection between the forward portion and the rearward portion of the band;

FIG. 4 is a view of a device to reduce, ease, or eliminate snoring similar to that shown in FIG. 3, with the addition of an air flow restrictor between the area of the mouth apertures and/or the insert and the remote apertures toward the sides;

FIG. 5 is a view illustrating how a device to reduce, ease, or eliminate snoring as shown in any of FIGS. 1-4 can be comfortably positioned and worn by a person, using the particular embodiment shown in FIG. 3 as an example;

FIG. 6 is a view illustrating how a device having an insert can be comfortably positioned in the mouth by a person, using the particular embodiment shown in FIG. 3;

FIG. 7 is a view of a device to reduce, ease, or eliminate snoring of a dog according to the first aspect of the invention, with the addition of cheek keepers; and

FIG. 8 is a view illustrating how a device to reduce or eliminate snoring as shown in the particular embodiment of FIG. 7 can be comfortably positioned to be worn by a dog.

### DETAILED DESCRIPTION OF PREFERRED EMBODIMENTS

The present inventions will be described by referring to apparatuses and methods showing various examples of how the inventions can be made and used. In these drawings, reference characters are used throughout the several views to indicate like or corresponding parts.

As used herein and in the appended claims, the words “comprise,” “has,” “having,” and “include” and all grammatical variations thereof are each intended to have an open, non-limiting meaning that does not exclude additional elements or parts of an assembly, subassembly, or structural element.

As used herein, terms such as “first,” “second,” “third,” etc. are arbitrarily assigned and are merely intended to differentiate between two or more parts that are similar or corresponding in structure and/or function. It is to be understood that the words “first” and “second” serve no other purpose and are not part of the name or description of the following terms. Furthermore, it is to be understood that the mere use of the term “first” does not require that there be any “second” similar or corresponding part, either as part of the same element or as part of another element. Similarly, the mere use of the word “second” does not require that there be any “third” similar or corresponding part, either as part of the same element or as part of another element, etc.

For the sake of consistency of usage, once a reference or relational term is arbitrarily assigned to help describe a structure or feature in a particular figure, the term will then be used consistently to refer to like parts throughout the other figures of the drawing. The same reference or relational term is later used even if the orientation of a structure is different in another figure. It is to be understood that, unless the context otherwise requires, the use of such arbitrarily assigned relational or relative terms is not to be construed as unnecessarily limiting the invention.

In general, unless otherwise expressly stated, the words or terms used in this disclosure and the claims are intended to have their ordinary meaning to persons of skill in the art. Initially, as a general aid to interpretation, the possible definitions of the words used herein are intended to be interpreted by reference to comprehensive general dictionaries of the English language published before or about the time of the earliest filing of this application for patent. Where several different general definitions are available, it is intended that the broadest definitions or senses be selected that are consistent with the description of the presently most-preferred embodiments of the invention, including without limitation as shown in the drawings.

After initially consulting such general dictionaries of the English language, it is intended that the words or compound terms be further defined or the most appropriate general definition or definitions be selected by consulting engineering dictionaries, encyclopedias, treatises, and relevant prior art to which this invention pertains. Finally, if necessary to resolve any remaining doubt, utilizing the patent record may be helpful to select from the possible definitions.

Of course, terms made up of more than one word (i.e., compound terms), such as “air passage,” may not be found in general dictionaries of the English language. Compound terms are intended to be interpreted as a whole and, as they would be understood in the art, consistent with the usage in this specification and with reference to the drawings.

It is intended that examining relevant general dictionaries, technical dictionaries, treatises, prior art, and the patent record will make it possible to ascertain the appropriate meanings that would be attributed to the words and terms of the description and claims by those skilled in the art, and the intended full breadth of the words and terms will be more accurately determined. In addition, the improper importation of unintended limitations from the written description into the claims will be more easily avoided.

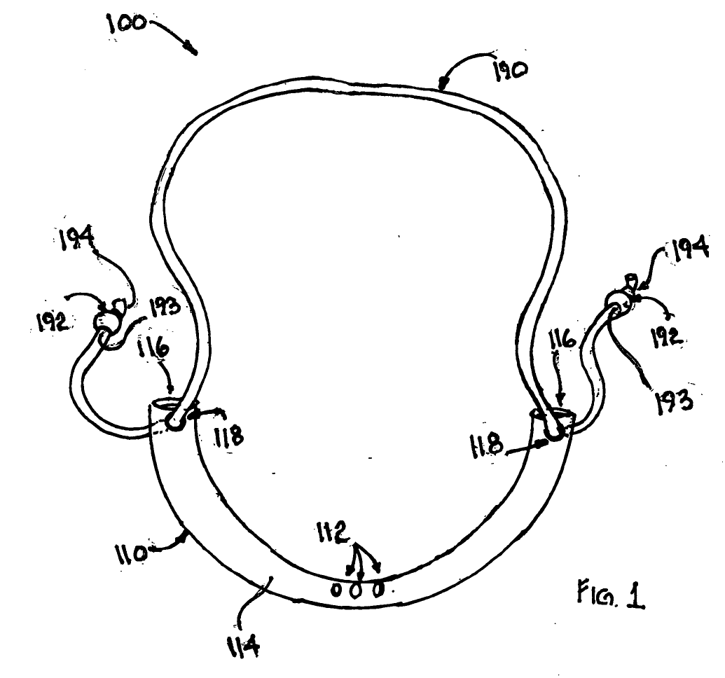

Referring now to FIG. 1 of the drawing, an anti-snoring device 100 according to the first aspect of the invention is illustrated. The device 100 is a band having a forward portion 110, preferably in the form of a tube, and a rearward portion 190, preferably in the form of an elastic strap.

Typically, the band is between 10-30 inches in circumference. Preferably, for use by a human adult, the band is about 24 inches in circumference. Preferably, the band is adapted to loosely fit around the head of a typical human adult.

The forward portion 110 of the band preferably has a substantially circular exterior cross section. More preferably, the forward portion 110 of the band has an outer diameter of about ½ inch. Preferably, the forward portion 110 of the band has a length at least sufficient to be positioned across the full width of the mouth of a typical human adult. According to the presently most-preferred embodiment of FIG. 1, the forward portion 110 of the band has a length of about of about 8 inches.

Preferably, the forward portion 110 of the band is made of an airtight material. More preferably, the forward portion of the band is made of a plastic material. Most preferably, the plastic material is a polyvinyl plastic. The plastic is preferably resilient, and more preferably of a material that feels relatively comfortable against the skin.

In this embodiment, the forward portion 110 of the band has at least one, and preferably several, mouth air apertures 112. The mouth apertures 112 are located on the forward portion 110 of the band such that, when the forward portion 110 of the band is positioned across the mouth, the air apertures 112 are oriented toward the mouth. Preferably, each of the mouth air apertures is a substantially round hole. More preferably, the hole of the mouth air aperture has an internal diameter of about ⅛ inch.

The air apertures 112 communicate with the air passage 114 of the forward portion 110 of the band. Preferably, the air passage of the forward portion of the band has a substantially circular interior cross section. According to the presently most-preferred embodiment, the air passage of the forward portion of the band has an internal diameter of about 5/16 inch.

In this embodiment of the invention, the tube 110 is open ended, which open ends serve as remote air apertures 116 away from the mouth air apertures 112. The remote air apertures 116 are located on the band such that, when the forward portion of the band is positioned across the mouth, the remote air apertures are adjacent the cheeks. Preferably, when the forward portion of the band is positioned across the mouth and the mouth air aperture is oriented toward the mouth, the remote air apertures 116 are oriented in any direction away from the skin.

Preferably, the rearward portion 190 of the band is made of an elastic material having an ultimate resilient elongation of about 200%. Most preferably, the rearward portion 190 of the band comprises a fabric material. A fabric material is more comfortable and breathes better against the skin, especially if the material is laying between the skin and bedding.

Preferably, the forward portion 110 of the band and the rearward portion 190 of the band are not integrally formed. The forward portion 110 of the band and the rearward portion 190 of the band are operatively connected together at the respective ends of each portion. In this embodiment, for example, the forward portion 110 of the band has a retraining aperture 118 adjacent the ends thereof so that the ends of the elastic strap material of the rearward portion 190 of the band can be threaded through the retaining apertures 118. A knot 194 can be tied at each end of the elastic strap material of the rearward portion of the band, which by virtue of the size of the knot 194 prevents the rearward portion 190 from escaping the retaining apertures 118 of the forward portion 110 of the band.

More preferably, as shown in FIG. 1, beads 192 can be used to help keep the ends of the elastic strap material of the rearward portion of the band connected to the ends of the forward portion 110 of the band. Each bead has a bore 193 therethrough and a larger outer dimension than the size of the retaining aperture 118. The beads 194 can be threaded onto the elastic strap material of the rearward portion 190 of the band. The size of the beads 192, when tied between the retaining apertures 118 and the knots 194, respectively, ensures that the rearward portion 190 of the band cannot easily escape through the retaining apertures 118.

Still more preferably, the bore 193 of each of the beads 192 is adapted to provide a relatively tight fit for the elastic strap material of the rearward portion 190 of the band. This resists the elastic strap material from easily slipping through the bore 193. But the fit is not so tight as to prevent the elastic strap material from being pulled through the bore by gripping the elastic strap material and the bead with a person's hands and forcing the bead and the strap material to move relative to one another, i.e., by sliding the bead on the elastic strap material or pulling the elastic strap material through the bead 192. Thus, the knot 194 and the bead 192 can be separated a desired distance. Because the tight fit of the elastic strap material through the bore 193 of the bead, which does not easily slip, manually adjusting the position of the bead 192 on the elastic strap material can be used to adjust the effective length of the rearward portion 190 of the band between the retaining apertures 118 of the forward portion 110 of the band. This provides the additional benefit of making the overall circumference of the band adjustable.

As will hereinafter be described in detail, the device 100 is adapted to be positioned around the head, wherein the forward portion 110 of the band is adapted to be positioned across the mouth, and the rearward portion 190 of the band is adapted to be positioned around the back of the head or neck. It is possible to breath around the forward portion of the band or through the mouth apertures 112, which communicate with the air passage 114 and through the air passage 114 to the remote air apertures 116.

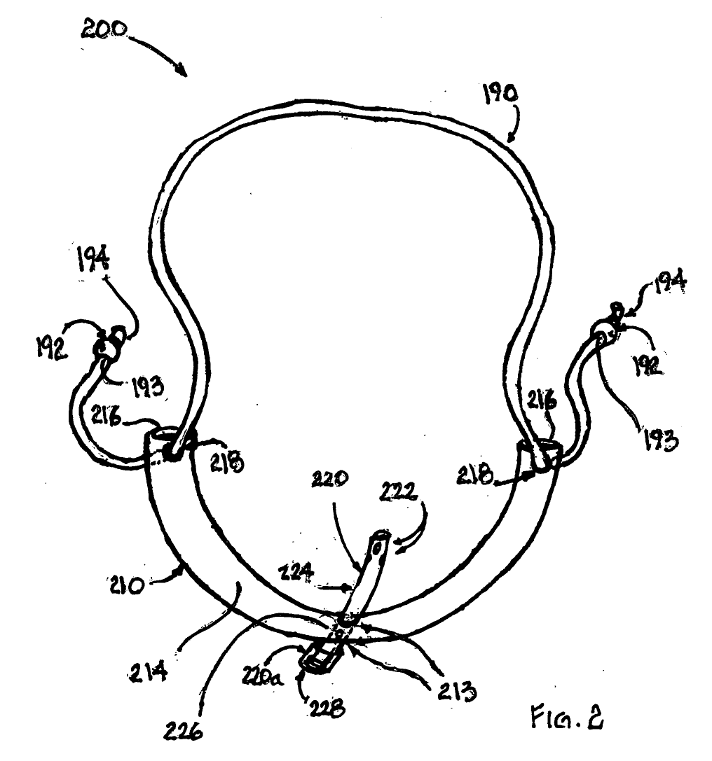

FIG. 2 is a view of a device 200 to reduce, ease, or eliminate snoring according to the third aspect of the invention. In general, the device 200 is similar to the device 100 described above, except as discussed and shown. The device 200 is a band having a forward portion 210, preferably in the form of a tube, and a rearward portion 190, preferably in the form of an elastic strap.

The forward portion 210 is similar to the forward portion 110 previously described above, but in this embodiment the device 200 does not have the mouth apertures 212. In this embodiment, the device 200 has at least one insert aperture 213 for an insert 220, as will hereinafter be described in detail. The forward portion 210 has an air passage 214. In this embodiment of the invention, the forward portion 210 is open ended, which open ends serve as remote air apertures 216.

The forward portion 210 of the band and the rearward portion 190 of the band are operatively connected together at the respective ends of each portion. In this embodiment, the connection is the same as previously described for the embodiment shown in FIG. 1. The forward portion 210 of the band has a retraining aperture 218 adjacent the ends thereof so that the ends of the elastic strap material of the rearward portion 190 of the band can be threaded through the retaining apertures 218. A knot 194 can be tied at each end of the elastic strap material of the rearward portion of the band, which by virtue of the size of the knot 194 prevents the rearward portion 190 from escaping the retaining apertures 218 of the forward portion 210 of the band.

More preferably, as shown in FIG. 2, beads 192 are used, each having a bore 193 therethrough, as previously described with respect to the embodiment shown in FIG. 1.

Continuing to refer to the embodiment shown in FIG. 2, an insert 220 is connected to the forward portion of the band, for example through the insert apertures 213. As will hereinafter be shown in detail, the insert 220 is adapted to be positioned to extend into the mouth between the tongue and hard palate but not so far as to contact the posterior pharynx. Preferably, the insert does not extend into the mouth beyond the hard palate. For example, preferably the insert does not extend into the mouth more than about 2 inches. More preferably, the insert 220 does not extend into the mouth more than about 1.5 inches.

The insert 220 is preferably tubular, having a substantially circular exterior cross section. Preferably, the insert has an outer diameter of about ½ inch.

In addition, the insert 220 preferably has at least one insert air aperture 222 adapted to be positioned in the mouth and has an air passage 224 through at least a portion of the insert 220. Preferably, the air passage of the insert has a substantially circular interior cross section. More preferably, the air passage 224 of the insert 220 has an internal diameter of about 3/16 inch.

The air passage 224 communicates with the air passage 214 of the forward portion 210 of the band. For example, the insert 220 can have a communicating aperture 226. Preferably, the air passage 224 of the insert extends the entire length of the insert. Most preferably, however, the end of the air passage 224 of the insert 220 that communicates between the air passage 224 of the insert and the air passage 214 of the forward portion of the band is otherwise closed.

According to the example of this preferred embodiment, the insert 220 extends completely through the forward portion of the band. In this embodiment, the two insert apertures 213, which are preferably circular holes co-axially located through the forward portion 210 of the band, are oriented so that the insert 220 is positioned to extend inward into the mouth, and an extending portion 220a protrudes from the other side of the forward portion 210 of the band. The insert 220 is preferably in the form of a tube, and the insert apertures 213 are adapted so that the insert 220 can be positioned therein but not easily slip therethrough. Preferably, there is an interference fit between the insert 220 and at least one of the insert apertures to help retain the insert connected to the forward portion 210 of the band.

Further, a cap retainer 228 is preferably positioned on the extending portion 220a. The retainer 228 is on the end of the insert that extends through the forward portion of the band, whereby the retainer prevents withdrawal of the insert from the forward portion of the band into the mouth. The retainer 228 has a larger outer cross section than the second aperture of the forward portion of the band. The cap retainer 228 also plugs or closes the outwardly extending portion 220a.

As will hereinafter be described in detail, the device 200 is adapted to be positioned around the head, wherein the forward portion 210 of the band is adapted to be positioned across the mouth, and the rearward portion 190 of the band is adapted to be positioned around the back of the head or neck. It is possible to breath around the forward portion of the band or through the insert 220, which communicate with the air passage 214 and through the air passage 214 to the remote air apertures 216.

Without being limited by any theoretical explanation, it is believed that the insert 220 helps keep the tongue separated from the roof of the mouth, which helps reduce the tendency to snore. It is also believed that the insert 220 is not so intrusive into the mouth as to cause a gag reflex.

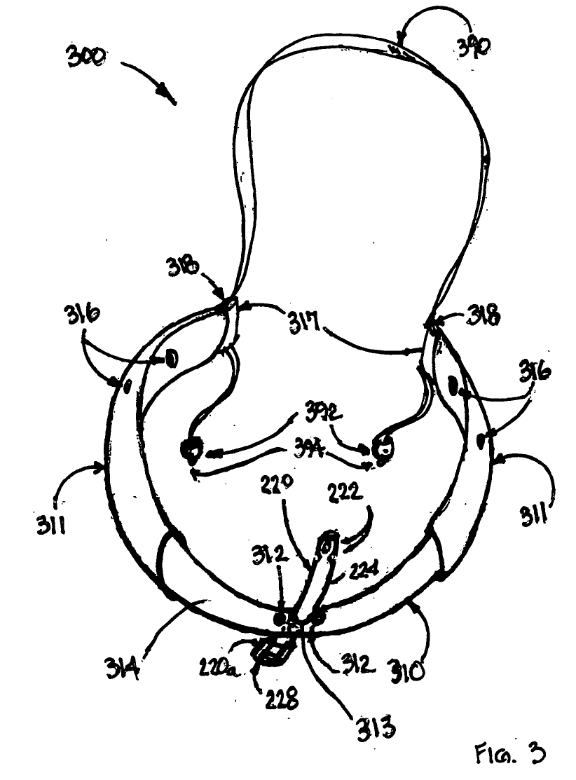

FIG. 3 is a view of a device 300 to reduce, ease, or eliminate snoring according to the second and third aspects of the invention, combined, and illustrating an alternative connection between the forward portion and the rearward portion of the band.

The device 300 is a band having a forward portion 310, preferably in the form of a tube, and a rearward portion 390, preferably in the form of an elastic strap. In this embodiment, the forward portion 310 has side portions 311 on the ends thereof. The side portions are slightly larger diameter tubular members having an inner diameter adapted to slip over the outer diameter of the forward portion 310. This provides an interference or “sticky” fit between the forward portion 310 and the side portions 311, particularly if they are both formed of plastic materials. While it is preferable for the forward portion 310 of the band to be formed of a relatively harder plastic material, the side portions 311, which are to be positioned around the head adjacent the cheeks, are preferably formed of a softer, more resilient plastic material that is more comfortable to wear or lie on.

In this embodiment, the forward portion 310 of the band has at least one, and preferably several, mouth air apertures 312. The mouth apertures 312 are located on the forward portion 310 of the band such that, when the forward portion 310 of the band is positioned across the mouth, the air apertures 312 are oriented toward the mouth. The air apertures 312 communicate with the air passage 314 of the forward portion 310 of the band.

In this embodiment, the device 300 also has at least one insert aperture 313 for an insert 220. The insert 220 has the same structure as previously described with respect to the embodiment shown in FIG. 2.

In this embodiment of the invention shown in FIG. 3, the side portions 311 have remote air apertures 316 away from the mouth air apertures 312. Preferably, the remote air apertures 316 are oriented on the band such that, when the forward portion 310 of the band is positioned across the mouth and the mouth air apertures 312 are oriented toward the mouth, the remote air apertures 316 are oriented outwardly and away from the skin. Preferably, the forward portion 310 of the band has a plurality of remote air apertures communicating with the air passage 314 therein, the plurality of remote air apertures being located on the band remote from the mouth. More preferably, at least one of the plurality of remote air apertures 316 is located on the band about 3 inches away from the mouth. Most preferably, the remote air apertures 316 are each a substantially round hole. In addition, the round hole of the remote air apertures preferably has an internal diameter of about ½ inch.

The forward portion 310 of the band and the rearward portion 390 of the band are operatively connected together at the respective ends of each portion. In this embodiment, for example, the side portions 311 on the forward portion 310 of the band each have a retraining connector 317 positioned in the end thereof so that the ends of the elastic strap material of the rearward portion 390 of the band can be threaded through the retaining apertures 318 provided by the retaining connectors 317. A knot 394 can be tied at each end of the elastic strap material of the rearward portion of the band, which by virtue of the size of the knot 394 prevents the rearward portion 390 from escaping the retaining apertures 318.

More preferably, as shown in FIG. 1, beads 392 can be used to help keep the ends of the elastic strap material of the rearward portion of the band connected to the retaining connectors 317. Each bead has a bore 393 therethrough and a larger outer dimension than the size of the retaining aperture 318.

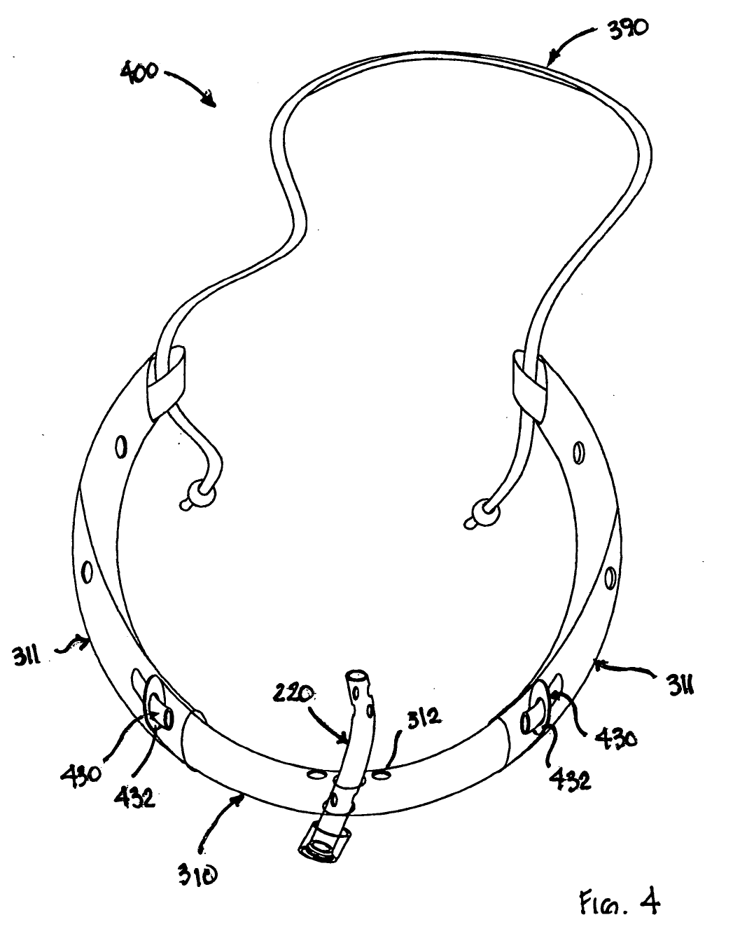

FIG. 4 is a view of a device 400 to reduce, ease, or eliminate snoring similar to that shown in FIG. 3, with the addition of an air flow restrictor 430 between the area of the mouth apertures and/or the insert and the remote apertures toward the sides. The air flow restrictor 430 can be, for example, a smaller inner diameter tubular member operatively positioned inside the forward portion of the band 310 or preferably inside the side portions 311, as shown. A spacer ring 432 can be used to help position the air flow restrictor 430.

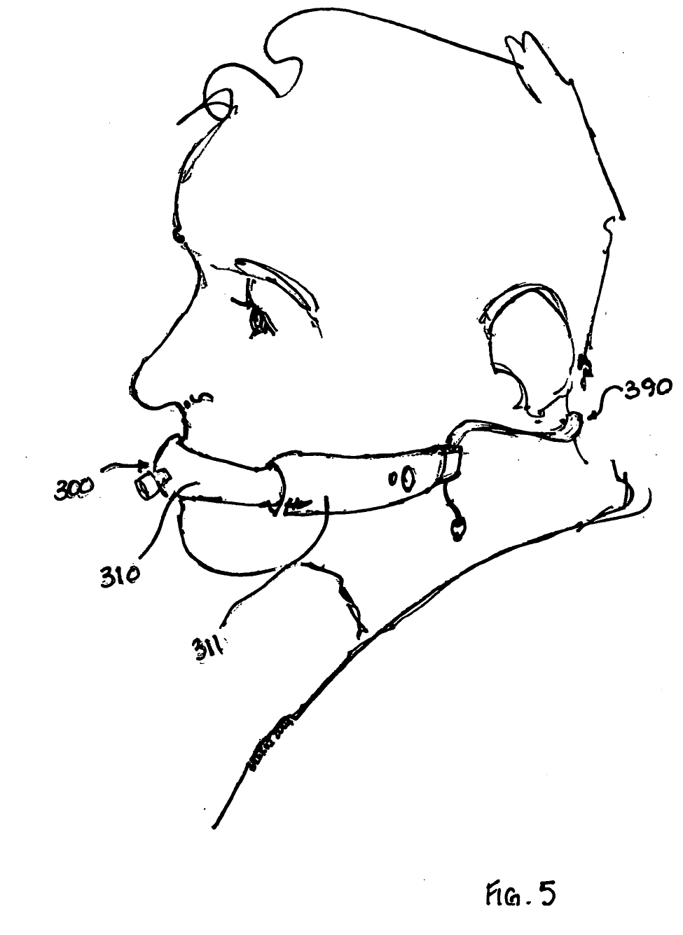

FIG. 5 is a view illustrating how a device to reduce, ease, or eliminate snoring as shown in any of FIGS. 1-4 can be comfortably positioned and worn by a person, using the particular embodiment of device 300 shown in FIG. 3 as an example. The device 300 is positioned around the head, with the forward portion 310 of the band positioned across the mouth, the side portions 311 of the forward portion 310 positioned across the cheeks, and the rearward portion 390 of the band positioned behind the head or neck.

Preferably, the forward portion of the band is adapted to be positioned between the upper and lower lips. Most preferably, the forward portion of the band is adapted to be positioned between the upper and lower lips and also at least slightly into the mouth.

Preferably, the rearward portion of the band is adapted to be positioned around the back of the neck and below the ears. More preferably, the rearward portion of the band is adapted to be loosely positioned around the back of the head or neck.

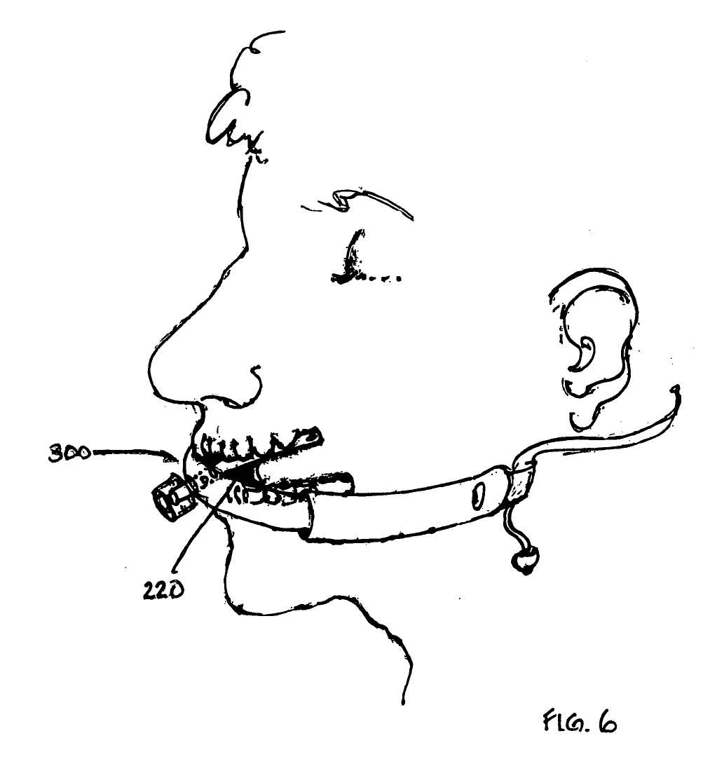

FIG. 6 is a view illustrating how a device having an insert 220 can be comfortably positioned in the mouth by a person, using the particular embodiment of device 300 shown in FIG. 3. The insert 220 of the device 300 is shown in the cut-away view of a person's mouth, which tends to keep the tongue separated from the hard palate of the mouth but the insert 220 does not extend so far into the mouth as to touch the pharynx, which might otherwise cause a gag reflex.

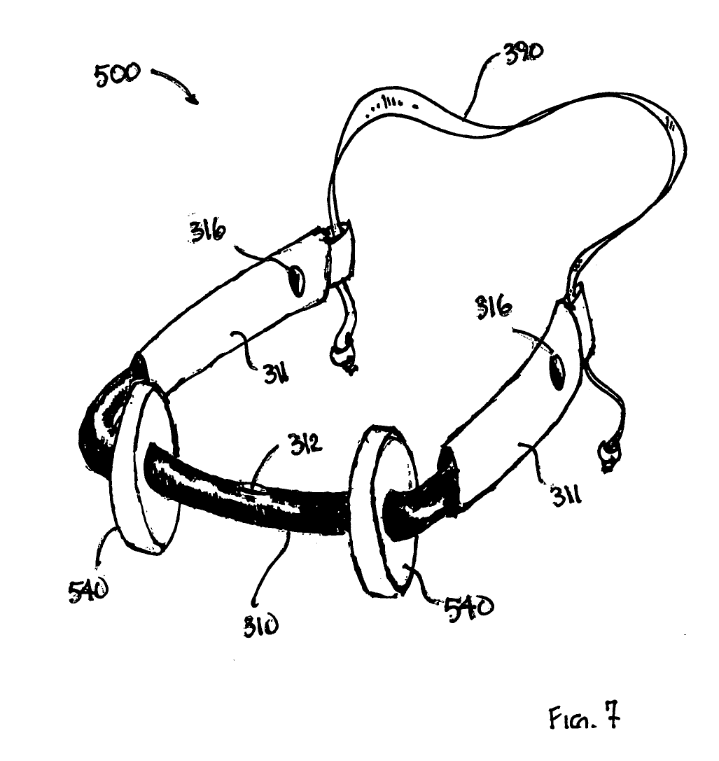

FIG. 7 is a view of a device 500 to reduce, ease, or eliminate snoring of a dog according to the first aspect of the invention, with the addition of cheek keepers 540. The device 500 is similar to the device 300 shown in FIG. 3, having a forward portion 310 with side portions 311 and a rearward portion 390 (but without an insert 220). For use with an animal, it is presently believed that it would often be better for the device to not include the insert 220, which the animal may not like. It is also believed, however, that an insert would still be beneficial for use in an anti-snoring device for an animal, if not objected to by the particular animal.

Preferably, as illustrated in FIG. 7, the forward portion of the band is adapted to better fit to go across the mouth of the animal for which the anti-snoring device is intended to be used. In the case of a dog, for example, it may be generally U-shaped and adapted to go between the lips of the dog and into the dog's mouth, between the tongue and hard palate.

The cheek keepers 540 are preferably in the form of disk-shaped plastic members that can be positioned on the forward portion 310 of the band.

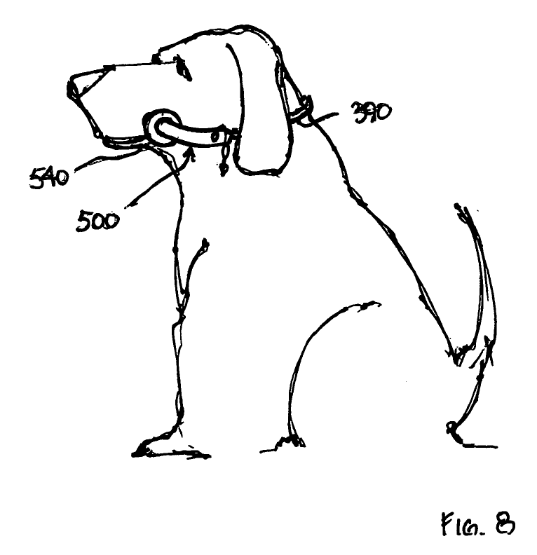

FIG. 8 is a view illustrating how a device to reduce or eliminate snoring as shown in the particular embodiment of FIG. 7 can be comfortably positioned to be worn by a dog.

According to the foregoing description, methods of using the devices according to the invention are provided. A method of using the device comprises the steps of, in any order: positioning the forward portion of the band across the mouth; positioning the rearward portion of the band around and behind the head or neck; and positioning the insert to extend into the mouth between the tongue and hard palate but not so far as to contact the posterior pharynx.

The step of positioning the rearward portion of the band can further comprise the step of positioning the rearward portion of the band around the back of the neck and below the ears. These steps can be performed on oneself. According to another aspect of the invention, the steps are performed on an animal, such as a dog. When performed on a dog, the step of positioning the forward portion of the band across the mouth further comprises the step of positioning the band inside the mouth.

The invention is described with respect to presently preferred embodiments, but is not intended to be limited to the described embodiments. As will be readily apparent to those of ordinary skill in the art, numerous modifications and combinations of the various aspects of the invention and the various features of the preferred embodiment can be made without departing from the scope and spirit of the invention. It should also be understood, for example, that the function of a single structure described herein can sometimes be performed by more than one part, or the functions of two different structures can be performed by a single or integrally formed part. Especially from manufacturing and cost perspectives, it is preferred to design the storage device to minimize the number of parts. These costs include not only the costs associated with making the parts, but also the costs of assembly. Preferably, the fewest possible number of parts and steps required to manufacture and assemble the apparatus, the better. The invention is to be defined by the appended claims.

1. A device to reduce, ease, or eliminate snoring, the device comprising:
a. a band, wherein:
i. a forward portion of the band is adapted to be positioned across the mouth;
ii. a rearward portion of the band is adapted to be positioned around the back of the head or neck; and
b. an air passage through at least a portion of the length of the forward portion of the band, wherein the air passage of the forward portion of the band comprises:
i. at least one mouth air aperture communicating with the air passage located on the forward portion of the band such that, when the forward portion of the band is positioned across the mouth, the air aperture is oriented toward the mouth; and
ii. at least one remote air aperture communicating with the air passage located on the band remote from the mouth.

2. A device to reduce, ease, or eliminate snoring, the device comprising:
a. a band, wherein:
i. a forward portion of the band is adapted to be positioned across the mouth;
ii. a rearward portion of the band is adapted to be positioned around the back of the head or neck; and
b. an air passage through at least a portion of the length of the forward portion of the band, wherein the air passage of the forward portion of the band comprises:
i. at least one mouth air aperture communicating with the air passage located on the forward portion of the band such that, when the forward portion of the band is positioned across the mouth, the air aperture is oriented toward the mouth; and
ii. at least one remote air aperture communicating with the air passage located on the band remote from the mouth; and
c. an insert, wherein:
i. the insert is connected to the forward portion of the band; and
ii. the insert is adapted to be positioned to extend into the mouth between the tongue and hard palate but not so far as to contact the posterior pharynx.

3. A device to reduce, ease, or eliminate snoring, the device comprising:
a. a band, wherein:
i. a forward portion of the band is adapted to be positioned across the mouth;
ii. a rearward portion of the band is adapted to be positioned around the back of the head or neck;
b. an insert, wherein:
i. the insert is connected to the forward portion of the band; and
ii. the insert is adapted to be positioned to extend into the mouth between the tongue and hard palate but not so far as to contact the posterior pharynx;
c. an air passage through at least a portion of the length of the forward portion of the band, wherein the air passage of the forward portion of the band has at least one remote air aperture communicating with the air passage located on the band remote from the mouth; and
d. an air passage through at least a portion of the insert wherein the air passage of the insert:
i. has at least one insert air aperture adapted to be positioned in the mouth; and
ii. communicates with the air passage of the forward portion of the band.

4. The device according to claim 1, wherein the band is between 10-30 inches in circumference.

5. The device according to claim 4, wherein the band is about 24 inches in circumference.

6. The device according to claim 1, wherein the band is adapted to loosely fit around the head of a typical human adult.

7. The device according to claim 1, wherein the band has a circumference that is adapted to be adjustable.

8. The device according to claim 1, wherein the forward portion of the band is tubular.

9. The device according to claim 8, wherein the forward portion of the band has a substantially circular exterior cross section.

10. The device according to claim 9, wherein the forward portion of the band has an outer diameter of about ½ inch.

11. The device according to claim 8, wherein the forward portion of the band has a length at least sufficient to be positioned across the full width of the mouth of a typical human adult.

12. The device according to claim 8, wherein the forward portion of the band has a length of about of about 8 inches.

13. The device according to claim 1, wherein the air passage of the forward portion of the band has a substantially circular interior cross section.

14. The device according to claim 13, wherein the air passage of the forward portion of the band has an internal diameter of about 5/16 inch.

15. The device according to claim 1, wherein the forward portion of the band has a plurality of mouth air apertures communicating with the air passage therein located on the forward portion of the band such that, when the forward portion of the band is positioned across the mouth, at least one of the mouth air apertures is oriented toward the mouth.

16. The device according to claim 1, wherein the mouth air aperture is a substantially round hole.

17. The device according to claim 16, wherein the hole has an internal diameter of about ⅛ inch.

18. The device according to claim 1, wherein the remote air aperture is located on the band such that, when the forward portion of the band is positioned across the mouth, the remote air aperture is adjacent the cheek.

19. The device according to claim 1, wherein the remote air aperture is oriented on the band such that, when the forward portion of the band is positioned across the mouth and the mouth air aperture is oriented toward the mouth, the remote air aperture is oriented in any direction away from the skin.

20. The device according to claim 19, wherein the remote air aperture is oriented on the band such that, when the forward portion of the band is positioned across the mouth and the mouth air aperture is oriented toward the mouth, the remote air aperture is oriented outwardly and away from the skin.

21. The device according to claim 1, wherein the forward portion of the band has a plurality of remote air apertures communicating with the air passage therein, the plurality of remote air apertures being located on the band remote from the mouth.

22. The device according to claim 21, wherein the at least one of the plurality of remote air apertures is located on the band about 3 inches away from the mouth.

23. The device according to claim 21, wherein at least one of the remote air apertures is oriented on the band such that, when the forward portion of the band is positioned across the mouth and the mouth air aperture is oriented toward the mouth, the remote air aperture is oriented outwardly.

24. The device according to claim 1, wherein the remote air aperture is a substantially round hole.

25. The device according to claim 24, wherein the hole of the remote air aperture has an internal diameter of about ½ inch.

26. The device according to claim 1, wherein the forward portion of the band has a pair of spaced-apart keepers to help maintain the forward portion of the band between the cheeks.

27. The device according to claim 26, wherein the keepers are generally disk shaped.

28. The device according to claim 1, wherein the forward portion of the band is made of an air-tight material.

29. The device according to claim 28, wherein the forward portion of the band is made of a plastic material.

30. The device according to claim 28, wherein the forward portion of the band is made of plastic.

31. The device according to claim 30, wherein the plastic of the forward portion of the band is a polyvinyl plastic.

32. The device according to claim 28, wherein the forward portion of the band is made of a resilient material.

33. The device according to claim 32, wherein the forward portion of the band is made of a material that feels relatively comfortable against the skin.

34. The device according to claim 1, wherein the forward portion of the band is adapted to be positioned between the upper and lower lips.

35. The device according to claim 34, wherein the forward portion of the band is adapted to be positioned between the upper and lower lips and at least slightly into the mouth.

36. The device according to claim 1, wherein the rearward portion of the band is a strap.

37. The device according to claim 1, wherein the rearward portion of the band comprises an elastic material.

38. The device according to claim 37, wherein the elastic material has an ultimate resilient elongation of about 200%.

39. The device according to claim 1, wherein the rearward portion of the band comprises a fabric material.

40. The device according to claim 1, wherein the rearward portion of the band is adapted to be positioned around the back of the neck and below the ears.

41. The device according to claim 1, wherein the rearward portion of the band is adapted to be loosely positioned around the back of the head or neck.

42. The device according to claim 1, wherein forward portion of the band and the rearward portion of the band are not integrally formed.

43. The device according to claim 1, wherein forward portion of the band and the rearward portion of the band are directly connected to one another to form the band

44. The device according to claim 1, further comprising a connection between the ends of the forward portion of the band and the ends of the rearward portion of the band, respectively.

45. The device according to claim 44, wherein the connection between the forward portion of the band and the rearward portion of the band comprises: at least one aperture in each end of the forward portion of the band, whereby the ends of the rearward portion of the band can threaded through the apertures in the forward portion of the band, respectively.

46. The device according to claim 45, wherein the connection between the forward portion of the band and the rearward portion of the band further comprises: a knot in each end of the rearward portion of the band, whereby the ends of the rearward portions of the band are prevented from unthreading through the apertures in the forward portion of the band, respectively.

47. The device according to claim 46, wherein the connection between the forward portion of the band and the rearward portion of the band further comprises: at least one bead with an aperture therethrough, the bead being larger than the aperture in one of the ends of the forward portion of the band, whereby an end of the rearward portion of the band can be further threaded through the bead and positioned between one of the apertures in the forward portion of the band and one of the knots in the rearward portion of the band.

48. The device according to claim 44, wherein the connection between the forward portion of the band and the rearward portion of the band comprises: side portions interconnecting the ends of the forward portion of the band and the ends of the rearward portion of the band, respectively.

49. The device according to claim 48, wherein each of the side portions of the band are extensions of the forward portion of the band.

50. The device according to claim 49, wherein each of the extensions is connected to the ends of the forward portion of the band with an interference fit, respectively.

51. The device according to claim 50, wherein each of the ends of the forward portion of the band is tubular and each of the extensions is tubular.

52. The device according to claim 51, wherein each of the ends of the forward portion of the band fits into a hollow end of the extensions, respectively.

53. The device according to claim 52, wherein at least one of the extensions has an air passage therethrough communicating with the air passage of the forward portion of the band.

54. The device according to claim 53, wherein at least one of the extensions has a remote air aperture therein.

55. The device according to claim 1, wherein the insert does not extend into the mouth beyond the hard palate.

56. The device according to claim 1, wherein the insert does not extend into the mouth more than about 2 inches.

57. The device according to claim 1, wherein the insert does not extend into the mouth more than about 1.5 inches

58. The device according to claim 1, wherein the insert is tubular.

59. The device according to claim 58, wherein the forward portion of the band has an insert aperture adapted to accept a portion of the insert, whereby the insert can be connected to and extend into the forward portion of the band.

60. The device according to claim 59, wherein the forward portion of the band has a second insert aperture adapted to accept a portion of the insert, the second insert aperture located diametrically across the cross section of the forward portion of the band, whereby the insert can extend through the forward portion of the band.

61. The device according to claim 60, wherein there is an interference fit between the insert and at least one of the insert aperture and the second insert aperture to help retain the insert connected to the forward portion of the band.

62. The device according to claim 61, further comprising a retainer on the end of the insert that extends through the forward portion of the band, whereby the retainer prevents withdrawal of the insert from the forward portion of the band into the mouth.

63. The device according to claim 61, wherein the retainer has a larger outer cross section than the second aperture of the forward portion of the band.

64. The device according to claim 58, wherein the insert has a substantially circular exterior cross section.

65. The device according to claim 64, wherein the insert has an outer diameter of about ½ inch.

66. The device according to claim 1, wherein the insert has an air passage through at least a portion thereof, the air passage of the insert:
a. having at least one insert air aperture adapted to be positioned in the mouth; and
b. communicating with the air passage of the forward portion of the band.

67. The device according to claim 66, wherein the air passage of the insert extends the entire length of the insert.

68. The device according to claim 66, wherein the insert air aperture of the insert is positioned toward the end of the insert that is adapted to be positioned into the mouth.

69. The device according to claim 66, wherein the end of the air passage of the insert that communicates between the air passage of the insert and the air passage of the forward portion of the band is otherwise closed.

70. The device according to claim 66, wherein of the air passage of the insert has a substantially circular interior cross section.

71. The device according to claim 66, wherein the air passage of the insert has an internal diameter of about 3/16 inch.

72. A method of using the device according to claim 2, the method comprising the steps of, in any order:
a. positioning the forward portion of the band across the mouth;
b. positioning the rearward portion of the band around and behind the head or neck; and
c. positioning the insert to extend into the mouth between the tongue and hard palate but not so far as to contact the posterior pharynx.

73. The method according to claim 72, wherein the step of positioning the rearward portion of the band further comprises positioning the rearward portion of the band around the back of the neck and below the ears.

74. The method according to claim 72, wherein the steps are performed on oneself.

75. The method according to claim 72, wherein, when the steps are performed on a dog, the step of positioning the forward portion of the band across the mouth further comprises the step of positioning the band inside the mouth.
